(19) United States
(12) Patent Application Publication
(10) Pub. No.: US 2026/0207435 A1
(43) Pub. Date: Jul. 23, 2026

(54) SUPPLEMENT SYSTEM AND DEVICE

(71) Applicant: TESPO IP, LLC, Plymouth, MI (US)

(72) Inventors: Justin GAUVIN, Plymouth, MI (US); Ted Matthew MILLS, Plymouth, MI (US); Jacob WRIGHT, Plymouth, MI (US)

(73) Assignee: TESPO IP, LLC, Plymouth, MI (US)

(21) Appl. No.: 19/142,922

(22) PCT Filed: Jan. 5, 2024
(86) PCT No.: PCT/US2024/010525

§ 371 Date: Jun. 24, 2025

Related U.S. Application Data
(60) Provisional application No. 63/478,754, filed on Jan. 6, 2023.

(51) Int. Cl. A61J 7/04; A61J 1/03; A61J 7/00; G16H 20/13

(52) U.S. Cl. CPC A61J 7/0436; A61J 1/03; A61J 7/0069; G16H 20/13; A61J 2200/30; A61J 2205/60

(57) ABSTRACT

A system has a base member defining at least one chamber, and a cover cooperating with the base member to enclose the chamber. The cover includes at least one path associated with a respective chamber and positioned to be broken when the cover over the associated chamber is opened. A reader device has a housing sized to mate with a container enclosed by a cover, with the cover supporting a plurality of paths, with each path associated with and extending over each chamber. The reader device has first and second leads associated with a path of a respective chamber, and a controller configured to determine if a chamber is opened or unopened. A membrane for covering at least one chamber of a container is provided with a layer formed from an electrically non-conductive material, and a path formed from an electrically conductive material supported by the layer.

### CROSS-REFERENCE TO RELATED APPLICATIONS

This application claims the benefit of U.S. provisional application Ser. No. 63/478,754 filed Jan. 6, 2023, the disclosure of which is hereby incorporated in its entirety by reference herein.

### TECHNICAL FIELD

Various embodiments generally relate to a container and associated reader device for use in monitoring opening of at least one chamber of the container, and for use, for example, with a supplement such as a pharmaceutical and adherence tracking.

### BACKGROUND

Conventionally, consumers have supplements that are packaged in bulk in bottles, cans, or other containers. As such, the consumer has to remember the quantity and time that they would like to take the supplement. This may become more complicated for consumers that are taking multiple supplements simultaneously, as they may have different dosage or timing requirements associated with each supplement. This may also result in a consumer over-dosing or under-dosing. For a consumer that is ill, managing supplements may become complicated and difficult. Medical personnel, such as doctors, pharmacists, and other health professionals, may need to know if the consumer is taking their supplement, and if any doses have been missed, for example, for adherence tracking, for re-ordering, etc.

Pill boxes and the like have been developed to help organize pill consumption for people, but require a high degree of discipline to use regularly, and also require the person to manually fill the pill box with supplements on a regular basis as well as recall when to take the supplement.

### SUMMARY

According to one example, a system is provided with a base member defining at least one chamber, and a cover supported by the base member and cooperating with the base member to enclose the chamber. The cover includes at least one path, with each path associated with a respective chamber, and with each path positioned to be broken when the cover over the associated chamber is opened.

According to another example, a reader device is provided with a housing sized to mate with a container defining a plurality of chambers enclosed by a cover, with the cover supporting a plurality of paths, with each path associated with and extending over each chamber. The reader device has a first lead supported by the housing and extending outwardly therefrom, with the first lead positioned to be associated with the plurality of paths. The reader device has a plurality of second leads supported by the housing and extending outwardly therefrom, with each second lead positioned to be associated with a path of a respective chamber. A controller is supported by the housing and in communication with the first lead and each of the plurality of second leads, and is configured to (i) determine that one of the chambers in the container has been opened in response to a first electrical signal between the first lead and one of the second leads, and (ii) determine that another of the chambers is unopened in response to a second electrical signal between the first lead and another of the second leads.

According to yet another example, a membrane for covering at least one chamber of a container is provided. The membrane has one or more layers formed from an electrically non-conductive material, and one or more paths supported by the one or more layers. Each path extends from a first end to a second end, each path formed from an electrically conductive material.

### BRIEF DESCRIPTION OF THE DRAWINGS

FIG. 1 illustrates a perspective view of a reader device according to an embodiment;

FIG. 2 illustrates a perspective view of the reader device of FIG. 1 with a container;

FIG. 3 illustrates a perspective view of the reader device of FIG. 1 with a container and one opened chamber;

FIG. 4 illustrates a top view of a container with paths according to an embodiment;

FIG. 5 illustrates a top view of a pattern for paths according to various embodiments;

FIG. 6 illustrates a top view of a container with paths according to another embodiment;

FIG. 7 illustrates another top perspective view of the reader device of FIG. 1;

FIG. 8 illustrates a bottom perspective view of the reader device of FIG. 1;

FIG. 9 illustrates a cross-sectional schematic view of the reader device of FIG. 1;

FIG. 10 illustrates a cross-sectional schematic view of the reader device of FIG. 1 according to a first embodiment;

FIG. 11 illustrates a top view of a control board for use with the device of FIG. 10;

FIG. 12 illustrates a cross-sectional schematic view of the reader device of FIG. 1 according to a second embodiment;

FIG. 13 illustrates a top view of a control board for use with the device of FIG. 12;

FIG. 14 illustrates a perspective view of spring pins for use with the reader device of FIG. 1 and FIG. 10;

FIG. 15 illustrates a partial perspective view of the reader device of FIGS. 1 and 10;

FIG. 16 illustrates a top schematic view of a container with a membrane with NFC tags according to an embodiment, and for use with FIG. 1;

FIG. 17 illustrates a schematic view of a multipack container, such as a blisterpack, with paths according to an embodiment;

FIG. 18 illustrates a perspective view of a closed container with a reader device according to an embodiment; and

FIG. 19 illustrates a perspective view of the container of FIG. 18 with the reader device in an opened configuration.

### DETAILED DESCRIPTION

As required, detailed embodiments of the present invention are disclosed herein; however, it is to be understood that the disclosed embodiments are merely examples and may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure.

The invention relates generally to a reader device, container, membrane, and system for tracking opening of the container, for example, for adherence to a dosage schedule, or for tracking the date, time, and/or location of opening the container, e.g. to track opening and usage of the container contents; and may also provide connected adherence, tracking, customization, and guidance for the delivery and/or dosing of supplements.

As used herein, supplement may refer to a pharmaceutical, a dietary formula, a nutritional supplement, another edible, or any combination thereof. As used herein, supplement and formula or formulas are interchangeable. For example, a supplement may include a pill, a capsule, a caplet, a custom-formulated powder-form, a granulated and/or oil form, a beadlet-form, a liquid dietary formulas, and/or other pharmaceuticals or supplements in measured doses or any other ingestible material that may be fit into the serving chamber, a container, or as a standalone item. A supplement may also refer to other prescription or over the counter item such as an injectable medication, insulin, an ointment or other topical medication, medication for an inhaler, a liquid for use with a dropper such as an eye, ear, or nose dropper, a medication contained in a patch, or the like. A supplement may also refer to other consumables such as food or a liquid. Both terms are intended to broadly define any and all types of nutraceuticals, vitamins, minerals, fibers, fatty acids, proteins, amino acids, effervescents, herbal medicines, bodybuilding formulas, pharmaceuticals, therapeutics, medicines, pet products, drugs, treatments and any other like substance that is ingested or non-ingested for useful purposes. Formulas is also intended to broadly include foods for humans, animals and plants.

The supplements may be provided in various containers, such as a supplement dispensing pod, pill bottles, liquid bottles, blister packs, injectables, tubes, inhaler, eye dropper, ear dropper, nose dropper, patch, injectable pen, topical medicines, blister card, drinking bottle, food container, medical consumable, and other forms of packaging. In other examples, the container may be a shipping container or package, or may be a container with other contents and any number of chambers, including one chamber, or more than one chamber, and furthermore, may include a medical device, medical product, office product, office inventory, or other consumer product. For example, the container 104 may be packaging or a shipping box for a liquid ingestible, an alcoholic liquid beverage, liquid or dry soap, laundry detergent, dishwasher detergent, topical creams, wax substances, a wax candle, a mechanical component, electronics or an electronic component, jewelry or a gemstone such as a diamond or pearls, or other items.

A container 104 may have one or more chambers 106. In the example shown in FIG. 2, the container 104 has multiple chambers 106 that are separated from one another. Each chamber 106 has an associated opening that intersects a wall of the container 104. In the example shown, the container 106 additionally has a central opening to receive and mate with the reader device 100. In other examples, the reader device and container may be otherwise shaped or connected.

The container 104 has a cover 108, described in further detail below. The cover may overlay or connect with the container 104 to enclose each chamber 106, and furthermore, may seal the contents of each chamber within the chamber and container. Each chamber may contain one or more supplement(s). In one example, the container has a series of chambers with one supplement of one type within each chamber. In other examples, the container has a series of chambers, with multiple supplements of different types within each chamber. For example, one chamber may contain different pills or other supplements that are meant to be taken on the same day and at the same time. In other examples, the container 104 may have a single chamber 106 and associated cover 108 with one or more supplements or other content(s).

The container 104 may communicate with a remote device, which may be a personal device of the consumer, such as a personal health device, e.g. an activity tracker such as a wearable device that measures heart rate, steps, calories burned, and the like. The remote device may alternatively be a server, or a remote computer or device accessible by a health professional, a health advocate, a patient, the business, or the consumer.

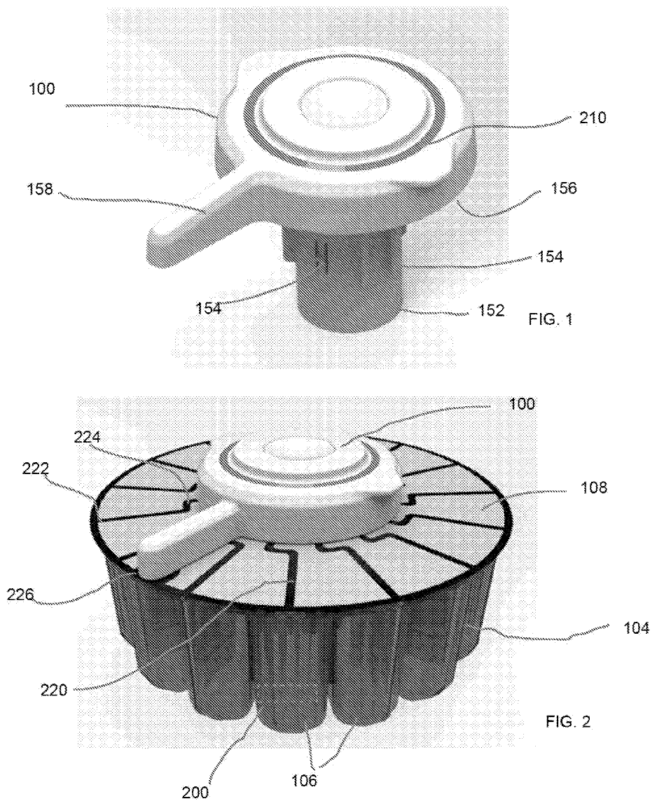

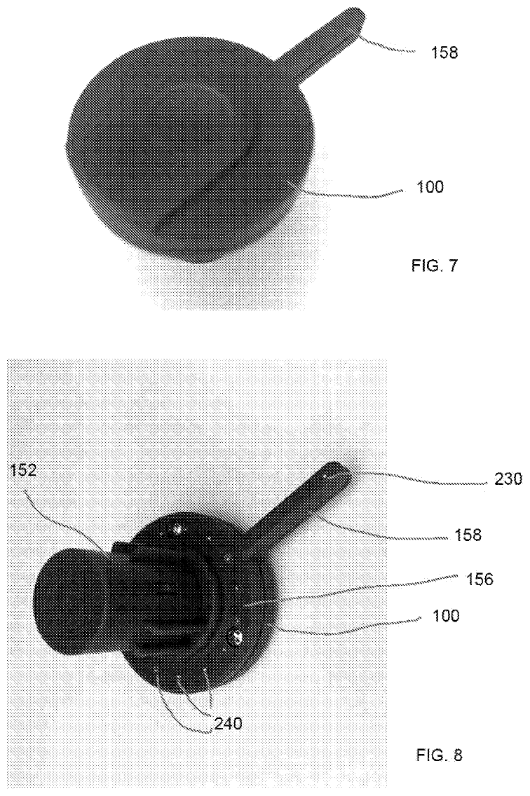

FIGS. 1-19 illustrate a reader device 100 and a container 104 according to various embodiments, and for use in monitoring opening or intake, improving adherence, and sending reminders to a user or a consumer. The reader device 100 may be used with a single user, or with multiple users, as described below. The reader device 100 may be a compact and portable design, and may be sized to mate with an individual container for portability and travel, or may be sized to sit on a countertop or table in a consumer's home. The reader device 100 may further be used without being connected to an external electrical source for true remote use via power from an internal battery.

The reader device 100 includes a controller and a transmitter or transceiver to connect and transfer information between different wireless protocols in order to transfer information to and from a consumer regarding scheduling of supplements, such as pharmaceuticals. The device 100 may further allow for communication of information to and from health professionals such as doctors, nurses, pharmacists, and the like. The device 100 is intended for home or personal use, and may aid the consumer in taking medication as prescribed, as well as providing information related to both patient dosage history and confirmation of supplements consumed and personal health information back to a server or remote health professional for use in medication reordering, changes in dosages or schedules, monitoring consumer patient health including whether a dose was taken, was taken on time, or was taken too often or too infrequently, and the like. The device may additionally be used to determine when a container is opened by a consumer, and may be used for containers or packages with any contents, for example, with shipping boxes or other containers.

The device 100 may further be provided with tag reading via an initiator 190, or data sensor 190, as described below to allow for use and tracking of containers that have a data tag 200. The data tag 200 may include information from the dispensing location, such as a pharmacy, and may be provided or programmed with a unique identifier or unique ID number, as well as information associated with the supplement within the container 104, such as name, dosage, quantity, and the like. The consumer receives the tagged container 104 from the pharmacy, with the pharmacy programming the tags. Alternatively, a health professional or other supplier of supplements may tag items or containers for the consumer, and furthermore may program the tags with information related to the supplement, container, dosage, or other information, or with information related to the personal item, via an application or other program. The tag may contain other information for non-pharmaceutical containers 104, including the contents, the intended recipient, and the like. The data tag 200 may be provided by a near-field communication (NFC) tag, an RFID tag, a UHF tag, a wired ID chip, a QR code, a bar code, a three-dimensional bar code, a beacon, or the like. The initiator 190 is provided as a sensor to read to detect the type of data tag 200 on the container. The data tag 200 may be provided on an upper surface, e.g. on the cover 108, or on another surface of the container 104, such as a lower surface as shown in FIG. 2.

The device 100 may additionally include a biometric sensor, that may be incorporated or located adjacent to sensor 190. The controller 160 is in communication with the biometric sensor to verify a user or consumer, and validate a user for the associated container, e.g. via fingerprint identification.

The device 100 may be wirelessly connected to a remote device or network via the transmitter or transceiver 180, and furthermore, may be connected to the internet via a wireless connection. In one example, the device 100 is connected via a wireless cellular connection, and in other examples, may be alternatively or additionally connected via a WiFi connection over a local area network such as 802.11. The device 100 additionally connects to other personal health devices, supplement dispensing machines, or the like via a wireless connection directly between the two devices, e.g. over Bluetooth.

The device 100 may provide a user output via an audio alert or notification, a visual alert or notification, e.g. via lights, and/or a haptic notification, e.g. via vibrations from a vibrator, to the user regarding a time to take medication, or the dose time. The controller 160 may control the audio or visual alerts based on information provided on the data tag 200. The controller 160 may further control lights to indicate which chamber is to be opened, and this may be based in part on information provided on a data tag 200. In other examples, the device 100 may be provided with a display screen to display messages, e.g. as a text-based message. FIG. 1 illustrates different lighting indicating that it is time to open a chamber, and which chamber is to be opened.

The device 100 may provide commands or notifications or alerts directly to the consumer. The apparatus 100 may be provided a transmitter or transceiver 180 for wireless communication over WiFi (via a local area network) and/or cellular network to communicate with other remote devices, such as remote servers or remote health professionals. The device 100 may also be provided with Bluetooth or similar wireless protocols to communicate with other paired devices, such as a personal mobile device or cellular phone of the consumer, paired supplement dispensers, or with paired personal health devices. The device 100 may additionally be provided with an initiator 190, such as a near field communication (NFC) device or other sensor to read tags, e.g. tags on a container 104, and may be further configured to write to other NFC devices in an active system. As used herein, a personal mobile device refers to a cellular telephone, a tablet, a personal computer such as a laptop or desktop computer, or the like.

In one example, the device 100 may be used in conjunction with or incorporated into a dispensing device and associated containers or pods 104, such as a device described in PCT Patent Publication No. WO 2015/073402, on May 24, 2015, or PCT Patent Publication No. WO 2016/126904, published Aug. 11, 2016, the entire disclosures of which is incorporated by reference herein, and for which copies were appended to and filed with priority U.S. provisional application Ser. No. 63/478,754. Furthermore, the device 100 may be used in conjunction with an application on a mobile device or computer of the consumer that may also provide notifications and alerts directly to the user. The device 100 may be provided with a lance or other device to assist in opening each chamber 106, and the lance may be supported by the housing 150 of the device, with the controller 160 controlling a position of the lance relative to the container 104 to selectively open a chamber by rupturing or otherwise removing the cover or membrane.

Referring to FIGS. 1-3 and 7-15, a device 100 is illustrated according to various embodiments. The device 100 has a housing 150 or base member 150. The housing 150 may be formed from a plastic material, and furthermore, may be formed using an injection molding technique or the like. In one example, the housing 150 is formed from at least two housing members that cooperate with one another to enclose a cavity defined by the housing 150.

In the example shown, the housing forms a central cylindrical member 152 that receives the battery. The central member 152 may be received by and cooperate with a central aperture of a container 104. The central member may further define splines or other locating and retention features 154 to engage the container 104.

The housing 150 also has an annular surface 156 surrounding the central member 152 and positioned to face the cover 108 when the device 100 is connected to a container 104. The housing 150 may additionally be provided with a radial arm 158 that extends outwardly from the annular surface 156 and central member 152. The radial arm also is positioned to face the cover 108 when the device 100 is connected to a container 104.

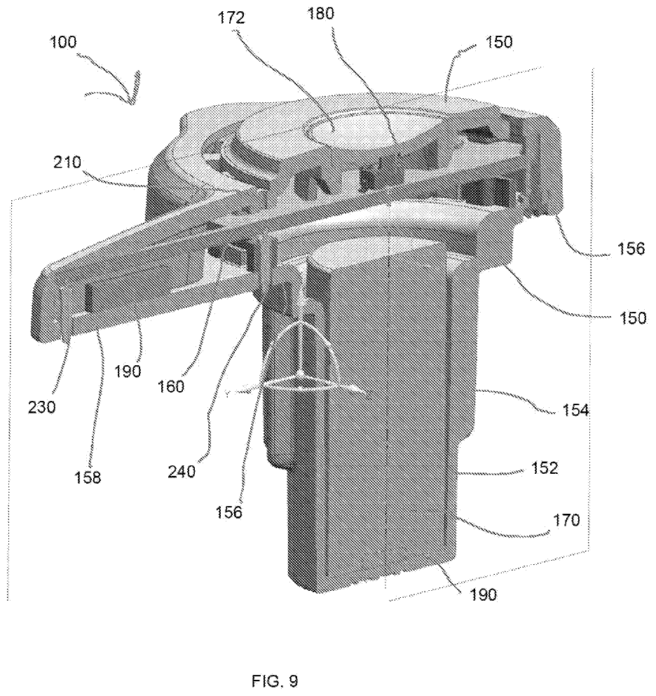

The housing 150 supports a controller 160, which may be provided on front and/or back sides of a board supporting the controller as shown, may be provided as one or more controllers or control modules for the various components and systems. The controller 160 and control system may include any number of controllers, and may be integrated into a single controller, or have various modules. Some or all of the controllers may be connected by a controller area network (CAN) or other system. It is recognized that any controller, circuit or other electrical device disclosed herein may include any number of microprocessors, integrated circuits, memory devices (e.g., FLASH, random access memory (RAM), read only memory (ROM), electrically programmable read only memory (EPROM), electrically erasable programmable read only memory (EEPROM), or other suitable variants thereof) and software which co-act with one another to perform operation(s) disclosed herein. In addition, any one or more of the electrical devices as disclosed herein may be configured to execute a computer-program that is embodied in a non-transitory computer readable medium that is programmed to perform any number of the functions as disclosed herein. In the example shown in FIG. 9, the controller 160 and many of the components of the apparatus 100 are provided on a single board mounted within the cavity of the housing.

The housing also supports a battery 170, which may be a rechargeable battery, in electrical communication with the controller. The battery 170 may be connected via a charging cable to recharge the battery via a remote power source such as a wall outlet, and furthermore may be charged via capacitive charging. In other examples, the battery may be provided as a one-time use, disposable battery. The housing 150 may be provided with a power button or on/off button 172 for the device.

The housing 150 also supports one or more wireless receivers, transmitters, or transceivers 180. In one example, the apparatus has a local area network (wireless or WiFi) transceiver, e.g. 802.11 b/g 2.4 Ghz, a cellular transceiver, e.g. 802.15 LTE Cat M1/Cat N61/EGPRS, or the like. The transceivers 180 allow for the controller to send and receive communications from the apparatus in a wireless manner. The housing 150 may further support a Bluetooth (BLE) transceiver that allows for communication with paired devices, such as a paired personal mobile device or telephone of the consumer, or a paired personal health device.

The housing 150 may additionally support a data sensor 190, such as a near field communications (NFC) initiator 190 or other sensor as described herein, to read a data tag on a container 104. Although the initiator 190 is shown on the arm, the initiator may alternatively be located elsewhere in the housing, including on the end region of the battery compartment to be adjacent to the other side of the container 104. The initiator 190 may detect an NFC, UHF, or passive high frequency RFID (or HF-RFID) tag 200, for example, as a tag or target that is provided and programmed on or in one or more recordable containers or personal items such as supplement dispensing pod, bottle, container, inhaler, eye dropper, ear dropper, nose dropper, patch, wrist band, necklace, ring, injectable pen, topical medicines, blister card, drinking bottle, food container, medical device, medical product, medical consumable, office product, office inventory, labeled cards, numbered cards, or the like, for example, via radio-frequency identification (RFID) by generating a radio frequency field. In other examples, the initiator 190 or sensor 190 may detect other data tags 200 as described herein. A data tag 200 may be attached to each item or container 104. Each tag 200 may be provided with a unique identifier to associate it with the container or personal item or supplement, and may further contain additional information as described herein. The tag may be provided with an adhesive backing or may otherwise be adhered to the container or personal item. Alternatively, the tag may be printed or otherwise incorporated into the item or container. Furthermore, the tag may be provided as a fob that is connected to the container or item, e.g. via a fastener such as a clip. The tag may alternatively be provided on a wristband or card and contain information regarding the identity of the consumer, and may be used by the apparatus 100 to confirm the identity of the consumer, for periodic requests for the consumer to check in, or the like. For an apparatus 100 intended for use with multiple users, each user may be required to confirm their identity to the apparatus 100 via an associated identifier tag, e.g. on a wristband, in response to the apparatus 100 calling them by name or providing a color light indicative of the associated user. The apparatus 100 may then provide customized instructions and or requests for information for that user, e.g. as to their supplement or other required information.

The apparatus 100 may additionally be provided with one or more indicator lights 210 that provides information, alerts, or notifications to the consumer. The indicator lights 210 may in communication with the controller 160 and controlled thereby. In one example, at least some of the indicator lights, e.g. the ones in the user interface region, may be mounted on the same board as the integrated circuit. The lights 210 may be provided by one or more light emitting diodes that are controller by the controller 160 to provide different color and/or patterns of light emission to provide notification.

The cover 108 may be provided as a membrane according to various non-limiting examples, and furthermore may be provided as a puncturable membrane. The cover 108 may be formed from one or more layers, and may include plastic, metal foil, paper, paint, and other layers. The cover may include an adhesive layer to connect the cover 108 to the container 104. In other examples, the cover 108 may be connected to the container 104 via other techniques such as sonic welding or the like. In one example, at least some of the layers of the cover 108 are electrically non-conducting, such as paint or plastic.

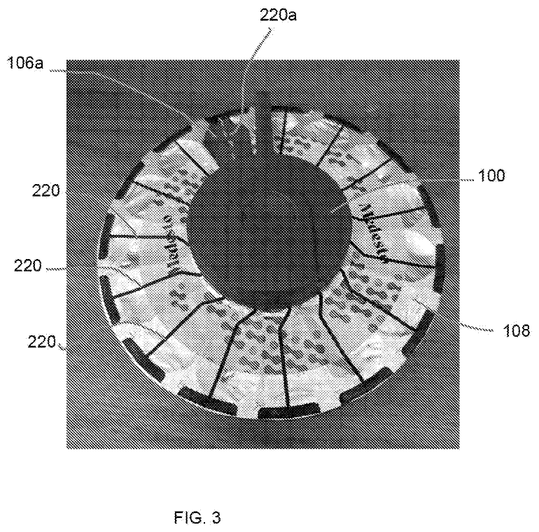

The cover 108 supports a plurality of paths 220. The cover may be provided with one path 220 that is associated with each chamber 106, respectively. In one example, each path 220 is positioned over or extends over the associated chamber 106. When a chamber of the container is opened, as is shown by chamber 106a in FIG. 3, the associated path 220a overlaying that chamber is also ruptured or broken.

The device 100 has at least one lead 230 that is supported by the housing 150, with the leads configured to interact with the paths to determine whether a path is intact and the associated chamber is unopened, or whether the path has been ruptured or otherwise divided and the associated chamber is opened, with the contents of the chamber presumably removed. The device 100 is therefore able to track which chambers have been opened, and provide data for dispensing of the contents of the container 104, for example, for adherence to supplement usage.

The controller 160 is in communication with the lead(s) 230 and configured to determine that a chamber has been opened based on signal(s) from the lead(s) 230. The device 100 may be configured to determine when a chamber is opened based on a change in the signals as described below. The device 100 may also be configured to determine which chambers are already open or closed when the device is connected to a container and turned on.

In various examples shown in FIGS. 2-5, the cover 108 has a plurality of paths 220 to correspond to the plurality of chambers 106, and each path extends from a first end 222 to a second end 224. The paths 220 may be formed from an electrically conductive material. In one example, the paths 220 may be printed onto the membrane 108 via conductive ink, and may be printed onto an outer surface of the layers of the cover 108. In various examples, the conductive ink may be provided by a silver-based ink or a carbon-based ink. Each path 220 may extend across or be located above an opening to the associated chamber 106. In other examples, the conductive paths 220 may be provided by conductive tape, and/or a conductive strip. The paths 220 are positioned to be broken at a location between their respective first and second ends 222, 224 when the cover 108 over the associated chambers 106 is ruptured.

Furthermore, the first ends 222 may be interconnected or directly connected to one another, for example, via a common electrode region 226 or touch point. The second ends 224 may be spaced apart from and separate from one another to provide an associated chamber-specific electrode region 228 or touch point.

The paths 220, including the first and second ends 222, 224 may be positioned radially outboard of the central aperture in the cover 108. The first ends of the paths 220 may be radially spaced apart from the second ends of the paths 220. The paths 220 may be provided with various shapes, including linear, or other complex shapes, including curves. By having a complex shaped path, the likelihood of the path 220 being ruptured when the cover is broken increases. The paths 220 may all be the same as one another, or may be different from one another as is shown in FIG. 5 to provide varying signals to the controller 160 depending on which path 220 is broken.

In the example shown, the device has a first lead 230, and a plurality of second leads 240. The leads 230, 240 are positioned to be associated with the paths 220 of a container 104. The first lead 230 may be provided by a pin, and is positioned at a distal end of the arm 158, with an end of the first lead 230 extending outwardly from the lower surface of the arm 158. The first lead 230 is positioned to contact the first common electrode region. In other examples, the device 100 may have a plurality of first leads 230 as well, with a separate pair of leads 230, 240 for each path.

The second leads 240 may also be provided as pins and are positioned radially about the annular surface 156, and extend outwardly from the annular surface 156. Each second lead 240 is positioned to contact a respective chamber specific electrode region.

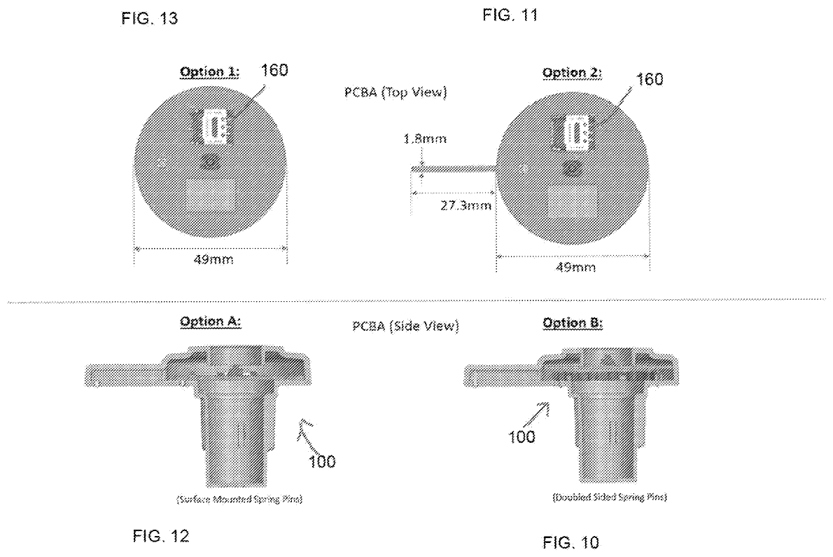

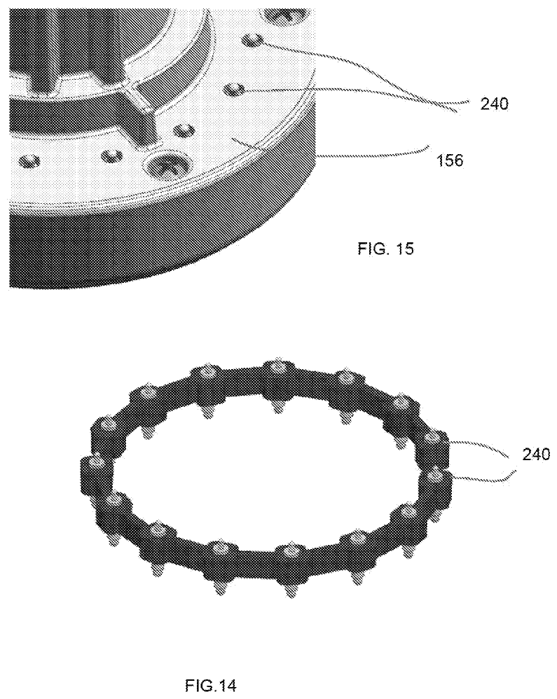

The first and second leads 230, 240 may be provided by spring-mounted pins. In one example, and as shown in FIG. 12, the first and second leads 230, 240 are provided by surface mounted spring pins, and with the pins mounted directly to the PCB, or to the housing 150 with a wired connected from a pin (such as the first pin) to the PCB. In another example, and as shown in FIGS. 10 and 14, the first and second leads 230, 240 are provided by double sided spring pins, and may be mounted onto a ring that is supported within the housing and with contact points on the PCB for the inner ends of the pins. The PCB may have an arm as shown in FIG. 11 to extend in the arm 158 of the housing 150, with an electrical connection such as an electrode path for the first lead 230 extending or printed down the arm, or the PCB may be provided without an arm as shown in FIG. 13 with a wired or other connection from the PCB to the first lead 230.

Of course, the first and second leads 230, 240 may be positioned otherwise for other containers, with a non-limiting example of such a container 104 shown in FIG. 17. Note that the paths 220 as drawn on the blisterpack of FIG. 17 are conceptual, and may be otherwise arranged.

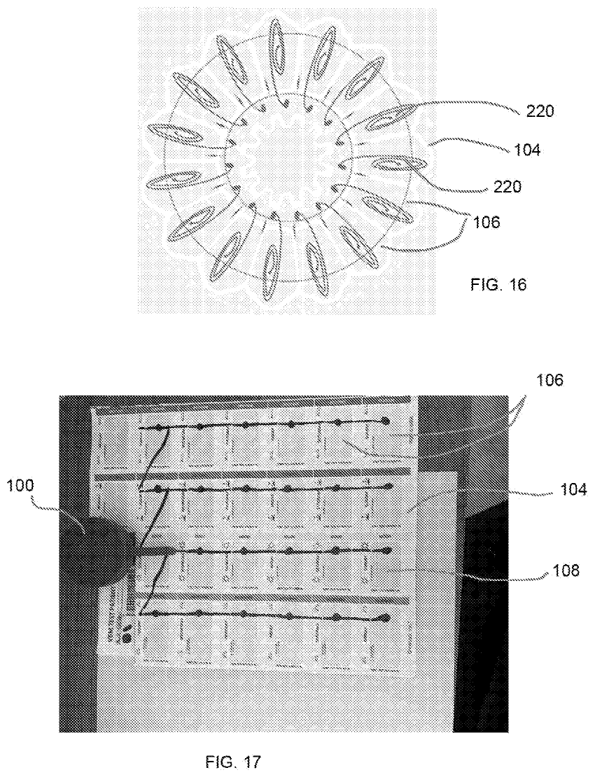

In other examples, the paths 220 may each be provided by a near-field communication coil tag as shown in FIGS. 6 and 16, and the one or more leads on the device provided by a tag reader similar to the initiator 190 described above. The tags may be separately applied to the cover as shown in FIG. 6, or may be printed or integrated in a layer of the membrane as shown in FIG. 16. In further examples, the paths 220 may be provided by magnetic materials, with the leads detecting a magnetic field. The controller 160 would determine that a chamber is open when the tag or path 220 is no longer readable.

The controller 160 is in communication with the first lead 230 and each of the plurality of second leads 240. The controller 160 is configured to (i) determine that one of the chambers 106 in the container has been opened in response to a first electrical signal between the first lead and one of the second leads, and (ii) determine that another of the chambers is unopened in response to a second electrical signal between the first lead and another of the second leads. The controller 160 measures an electrical signal across each path 220, e.g. as a voltage, current, or resistance, and can therefore determine when a chamber has been opened (with the path cut or broken) based on the change in or difference in the signals as determined from the first and second leads. When a path 220 is unbroken or intact, the path 220 acts to close a circuit between the first lead 230 and the associated second lead 240, or electrically connect the first and second leads. When the path 220 is broken, the circuit between the first and second paths is closed. In one example, the controller 160 sends a voltage at a specified current across a path 220 and measures across the path 220 via the leads 230, 240 to determine a change in the path 220, e.g. the conductivity or resistance.

For paths of different lengths or having different properties, the paths 220 may be arranged in a parallel configuration, and the device 100 and container 104 may be able to have a common touch point or electrode for the second ends of the paths as well, with the controller 160 determining which path was ruptured.

The controller 160 may then transmit which chambers 106 are opened via the transmitter 180 based on the signals from the first and second leads 230, 240. The controller 160 may additionally record and transmit a time stamp associated with the signal from the path 220 changing and when the chamber was opened.

The controller 160 may transmit information via the transmitter 180. For example, the controller 160 may transmit a signal indicative of dispensing of supplements and/or a time of dispensing based on a path being broken.

The controller 160 may provide a signal indicative of a user notification for a reminder for a user to take a supplement at a specified time in response to a signal being wirelessly received. The controller 160 may output the user notification via a user interface via a visual notification and/or an audible notification.

The controller 160 may further provide a notification to the user in response to receiving a signal, and this notification may be a request for the user to contact a provider, such as a doctor or a pharmacist, or for the user to review other information or notifications, e.g. messages from a provider.

The paths 220 may be used on a bottle, where the path is broken when the bottle is opened. The paths 220 may alternatively be used on packaging, such as a box or other shipping container, with the path 220 broken when the container is opened.

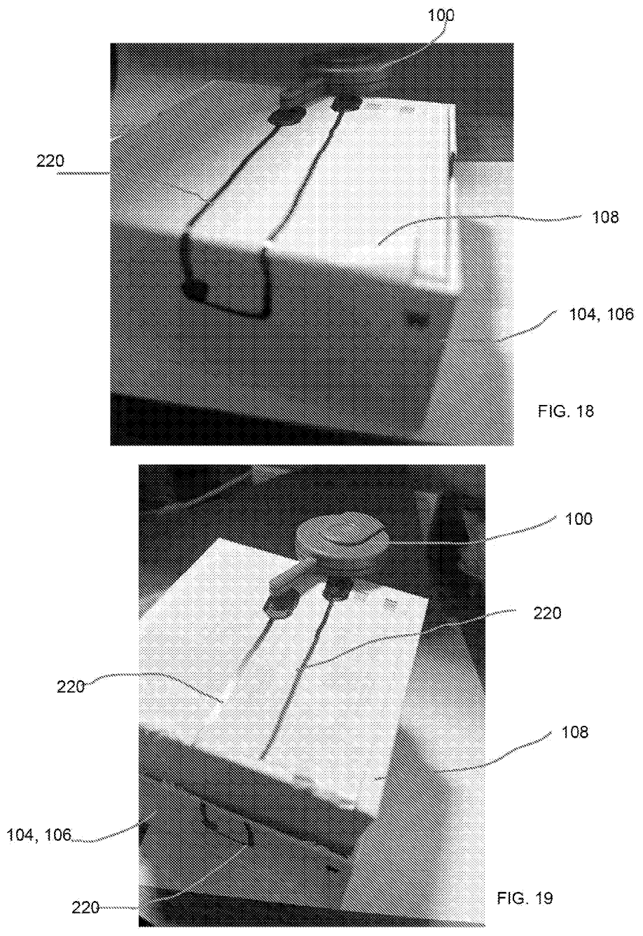

The paths 220 may be provided on a sticker that can be applied to containers of different shapes and/or sizes, with an example shown in FIGS. 18-19. In FIG. 18, the package is sealed with a single path 220 unbroken, and in FIG. 19, the package has been opened with the path 220 broken. The device 100 may stay with the package, and be disposable or reusable and returned. The device may additionally provide for tracking of the package, e.g. via a cellular or BLE transmitter, or via other geo-locating technology, including a global positioning system (GPS). The device 100 may be located within the container 104.

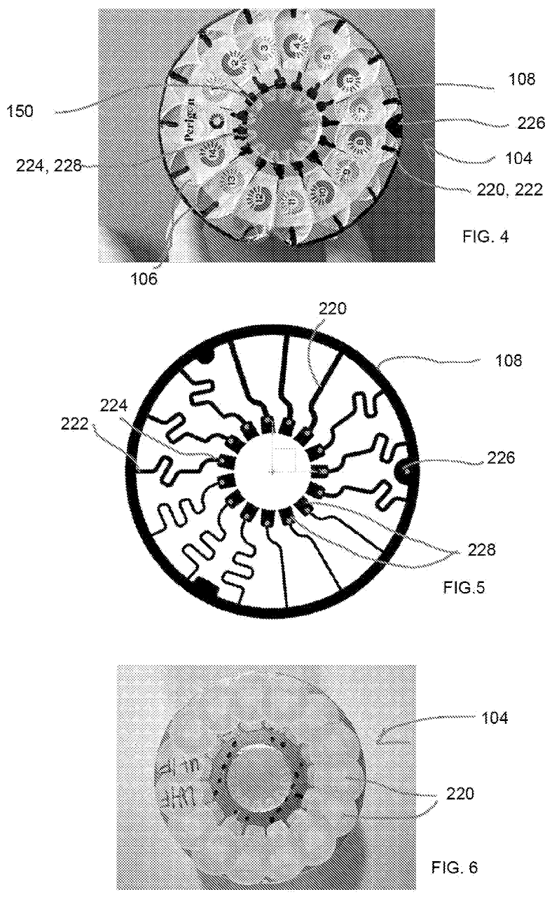

The paths 220 may be at least partially covered, as shown in FIG. 4, with labels or printing, to provide a visual indication of contents, for labeling purposes, graphics, or the like. This layer may be electrically non-conductive, and may be similar to a masking layer.

Various embodiments therefore relate to a container 104 that may provide security and tamper-proof or tamper-resistant features via the cover 108 and path(s) 220. The container and cover may be waterproof or water resistant, and furthermore, may be airtight. The container provides storage for supplements or other contents as described herein in one or more individual chambers of the container. The reader device 100, in conjunction with the path(s) on the cover 108 of the container 104 may provide for identification of an opening event of a chamber with known contents to provide and/or transmit information or confirmation of that the container was opened and the contents were dispensed from the associated chamber.

While exemplary embodiments are described above, it is not intended that these embodiments describe all possible forms of the disclosure or invention. Rather, the words used in the specification are words of description rather than limitation, and it is understood that various changes may be made without departing from the spirit and scope of the invention. Additionally, the features of various implementing embodiments may be combined to form further embodiments of the invention.

1. A system comprising:
a base member defining at least one chamber containing a supplement;
a cover supported by the base member and cooperating with the base member to enclose the chamber, the cover comprising at least one path, each path associated with a respective chamber, each path positioned to be broken when the cover over the associated chamber is opened; and
a reader device, the device comprising: a housing sized to mate with the base member, at least one lead associated with each path, and a controller in communication with the at least one lead, wherein the controller is configured to determine that one of the chambers is opened in response to receiving a signal from the associated at least one lead.

2. The system of claim 1 wherein each path extends from a first end, across an opening to the associated chamber, and to a second end; and
wherein the at least one path comprises conductive ink, conductive tape, and/or a conductive strip.

3-5. (canceled)

6. The system of claim 1 wherein the at least one path is a near-field communication coil tag.

7. The system of claim 1 wherein the cover comprises a puncturable membrane.

8. The system of claim 1 further comprising a data tag supported by the base member, wherein the data tag contains data indicative of a user name, contents of the at least one chamber, dispensing schedule, manufacturer information, and filling information.

9-13. (canceled)

14. The system of claim 1 wherein the reader device further comprises a data sensor configured to read a data tag, the sensor in communication with the controller.

15. The system of claim 14 wherein the reader device further comprises at least one light supported by the housing;
wherein the controller is configured to control at least one light supported by the housing in response to data from the sensor.

16-18. (canceled)

19. The system of claim 1 wherein the controller is configured to determine which chambers have been opened when the reader device is connected to the base member.

20. The system of claim 1 wherein the at least one chamber of the base member is a plurality of chambers, the cover extending over each chamber; and
wherein the at least one path is a plurality of paths, each path extending across an opening to an associated chamber when the cover covers the plurality of chambers of the base member.

21. The system of claim 20 wherein each path extends from a first end to a second end, wherein the first ends of the paths are connected to a common electrode, and wherein each second end is connected to an associated chamber-specific electrode; and
wherein each lead of the device comprises a first lead configured to contact the common electrode, and a second lead configured to contact a respective one of the chamber-specific electrodes.

22. (canceled)

23. The system of claim 1 wherein each path comprises a near-field communication coil tag; and
wherein each lead comprises a tag reader.

24. The system of claim 1 wherein the housing further comprises an arm extending outwardly from a central hub;
wherein a first lead of the at least one lead is supported by a distal end of the arm; and
wherein a second lead of the at least one lead is supported by the housing and spaced apart from the first lead.

25. The system of claim 24 further comprising a lance supported by the housing, the controller configured to control a position of the lance relative to the base member mated with the housing to selectively open one of the chambers by rupturing the cover and associated path.

26. A reader device comprising:
a housing sized to mate with a container defining a plurality of chambers enclosed by a cover, the cover supporting a plurality of paths, each path associated with and extending over each chamber;
a first lead supported by the housing and extending outwardly therefrom, the first lead positioned to be associated with the plurality of paths;
a plurality of second leads supported by the housing and extending outwardly therefrom, each second lead positioned to be associated with a path of a respective chamber; and
a controller supported by the housing and in communication with the first lead and each of the plurality of second leads, wherein the controller is configured to (i) determine that one of the chambers in the container has been opened in response to a first electrical signal between the first lead and one of the second leads, and (ii) determine that another of the chambers is unopened in response to a second electrical signal between the first lead and another of the second leads.

27. The device of claim 26 wherein each path extends from a first end, over the associated chamber, and to a second end;
wherein each second lead is positioned to contact only a second end of an associated path; and
wherein the first lead is configured to contact a common point in communication with first ends of the plurality of paths.

28-29. (canceled)

30. The device of claim 26 further comprising a sensor supported on the housing and positioned to read a data tag on a container, the sensor in communication with the controller; and
a user output supported by the housing, the controller configured to control the user output based on information provided by the data tag to indicate which chamber to open, wherein the user output further comprises at least one light, a speaker, and/or a vibrator.

31-33. (canceled)

34. The device of claim 26 wherein the housing further comprises an arm extending outwardly from a central hub;
wherein the first lead is supported by a distal end of the arm; and
wherein the plurality of second leads are positioned circumferentially about the central hub.

35. The device of claim 26 further comprising a lance supported by the housing, the controller configured to control a position of the lance relative to a container mated with the housing to selectively open one of the chambers by rupturing the cover and associated path.

36. (canceled)

37. The device of claim 26 further comprising a biometric sensor;
wherein the controller is configured to receive a signal from the biometric sensor, and validate a user for the associated container.

38. A membrane for covering at least one chamber of a container, the membrane comprising:
one or more layers formed from an electrically non-conductive material; and
one or more paths supported by the one or more layers, each path extending from a first end to a second end, each path formed from an electrically conductive material; each path comprises a conductive ink printed onto the one or more layers;
an adhesive layer attached to the one or more layers opposite to the one or more paths;
wherein the first ends of each path of the one or more paths are interconnected to one another, and the second ends of each path of the one or more paths are spaced apart from one another; and
wherein the first ends of the one or more paths are radially spaced apart from the second ends of the one or more paths.

39-44. (canceled)
